(19) United States
(12) Patent Application Publication
(10) Pub. No.: US 2019/0265258 A1
(43) Pub. Date: Aug. 29, 2019

(54) Method for Standardized Testing of Anandamide After Cannabinoid Consumption

(71) Applicants: Michele Reillo, Saint Augustine, FL (US); Howard Mann, Toronto (CA)

(72) Inventors: Michele Reillo, Saint Augustine, FL (US); Howard Mann, Toronto (CA)

(21) Appl. No.: 15/903,382

(22) Filed: Feb. 23, 2018

(51) Int. Cl. G01N 33/94; G01N 33/493; G01N 21/77; G01N 1/28; A61K 36/185; C07C 233/20

(52) U.S. Cl. CPC G01N 33/948; G01N 33/493; G01N 21/77; G01N 1/2813; G01N 2001/2826; C07C 233/20; G01N 2021/7759; G01N 2021/7786; A61K 36/185

(57) ABSTRACT

A method for standardized testing of anandamide after cannabinoid consumption quantifies the effectiveness of cannabinoid administration including all phytocannabinoids with and without tetrahydrocannabinol (T.H.C). Through the inclusion of a quantity of nitric oxide indicator, a user is able to determine a concentration of nitric oxide within a quantity of mammalian body fluid. The concentration of nitric oxide is an indication of the production of anandamide, a fatty-acid neurotransmitter that affects cannabinoid receptors within mammals. Cannabinoid receptors are involved with physiological processes including appetite, pain-sensation, mood, and memory.

### FIELD OF THE INVENTION

The present invention relates generally to pharmaceutical testing. More specifically, the present invention relates to a method for assessing the medicinal benefits for the consumption of cannabinoids.

### BACKGROUND OF THE INVENTION

Consumption of cannabis exemplifies potential medicinal benefits, as well as, recreational applications. Medicinal cannabis sometimes refers to cannabinoids, chemical compounds that act on cannabinoid receptors which are involved in physiological processes including appetite, pain-sensation, mood, and memory. While there are medicinal benefits for consuming cannabinoids, methods for measuring the benefits are not standardized which may lead to discrepancies between studies.

The present invention is a method for standardized testing of anandamide after cannabinoid consumption. Medicinally, the consumption of cannabis increases the brain's anandamide levels to help treat anxiety and depression. The present invention measures nitric oxide within a mammalian body fluid as the S stereoisomer of anandamide effects the production of nitric oxide in endothelial cells to quantify the benefits of cannabinoid consumption. The present invention makes use of a nitric oxide indicator to quantify the nitric oxide levels within the mammalian body fluid as the indicator changes color based on the concentration of nitric oxide within a mammal's body fluid. The color to which the indicator changes can be compared to a chromatic chart to indicate the level of nitric oxide production within the cannabinoid consumer's body. Therefore, the effectiveness of the cannabinoid consumption is able to be quantified. The present invention covers all types of cannabinoid administration including all phytocannabinoids with and without tetrahydrocannabinol (T.H.C).

### BRIEF DESCRIPTION OF THE DRAWINGS

FIG. 1 is a flow diagram depicting the steps of the present invention, wherein the quantity of mammalian body fluid is saliva.

FIG. 2 is a flow diagram depicting the steps of the present invention, wherein the quantity of mammalian body fluid is urine.

FIG. 3 is a schematic diagram for submerging the nitric oxide indicator within the quantity of mammalian body fluid

FIG. 4 is a schematic diagram for comparing the nitric oxide indicator with the chromatic identifier.

### DETAIL DESCRIPTIONS OF THE INVENTION

All illustrations of the drawings are for the purpose of describing selected versions of the present invention and are not intended to limit the scope of the present invention.

The present invention is a method for standardized testing of anandamide after cannabinoid consumption. The present invention quantifies the effectiveness of cannabinoid administration including all phytocannabinoids with and without tetrahydrocannabinol (T.H.C). Cannabinoid consumption increases anandamide, a naturally occurring fatty-acid neurotransmitter that affects cannabinoid receptors within mammals. Cannabinoid receptors are involved with physiological processes including appetite, pain-sensation, mood, and memory. Quantifying anandamide levels from the consumption of cannabinoids allows for the study of effectiveness of the cannabinoid consumption. The present invention is a method to standardize this quantification, as anandamide effects the production of nitric oxide within endothelial cells.

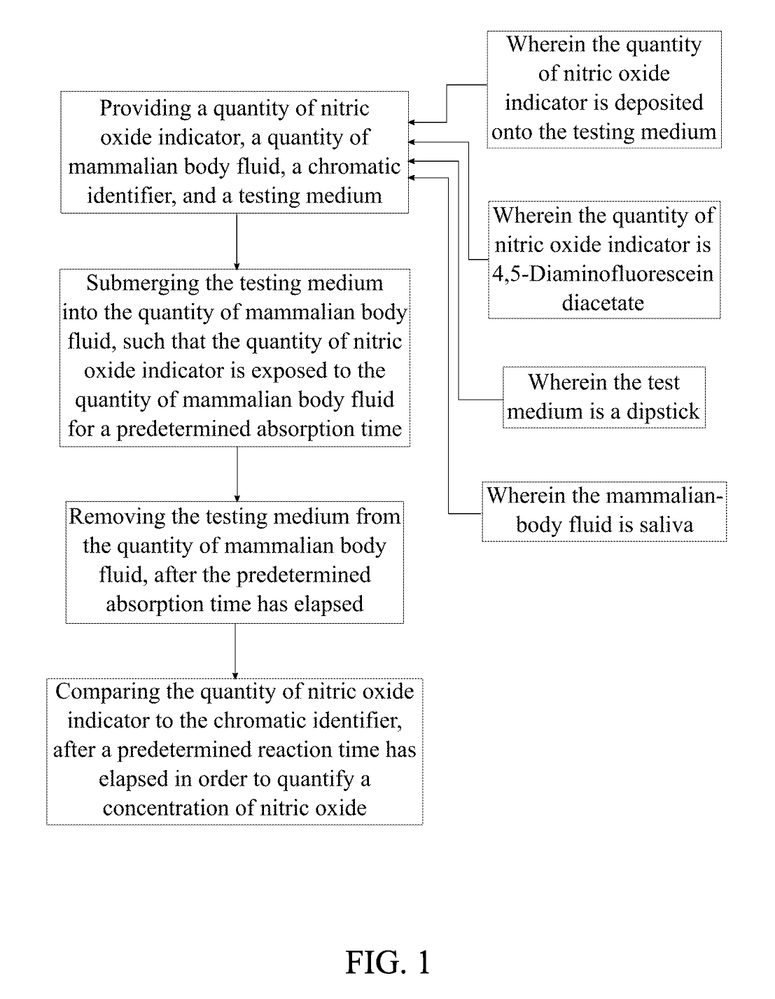

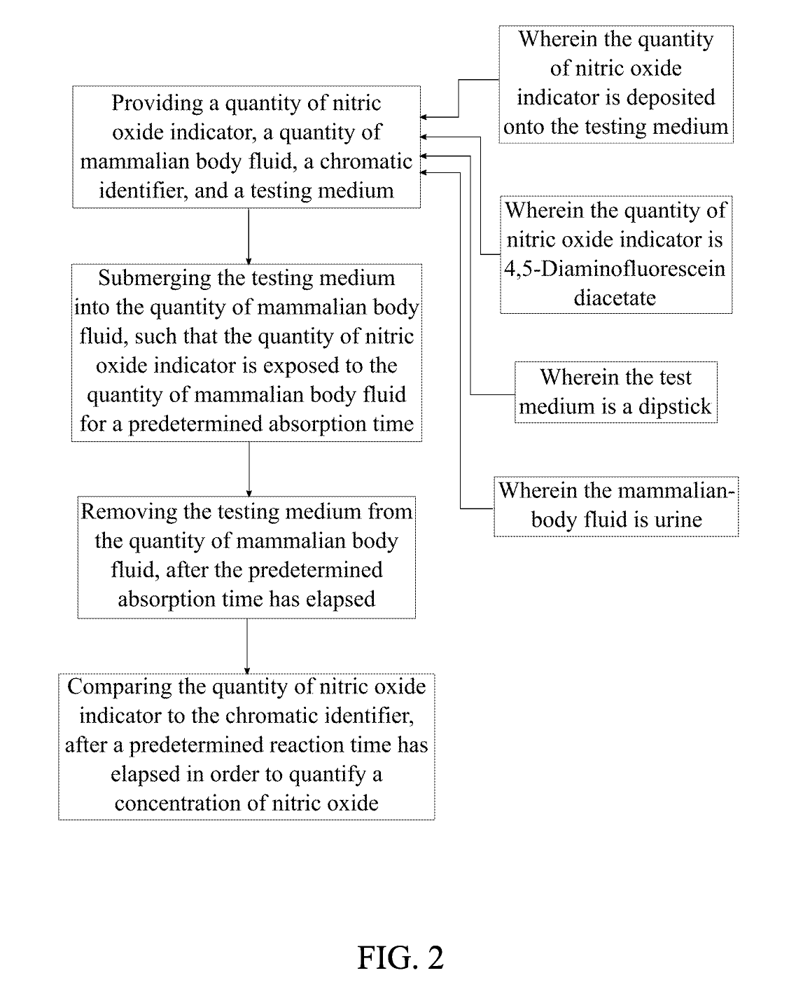

In order to execute the present invention, the present invention requires a quantity of nitric oxide indicator 1, a quantity of mammalian body fluid 2, a chromatic identifier 3, and a testing medium 4, wherein the quantity of nitric oxide indicator 1 is deposited onto the testing medium 4, as shown in FIG. 1. The nitric oxide indicator 1 is a fluorescent dye for nitric oxide detection. The nitric oxide indicator 1 is preferred to be 4,5-Diaminofluorecein diacetate, as 4,5-Diaminofluorecein diacetate is a cell permeable fluorescent detector of nitric oxide in living cells. The quantity of mammalian body fluid 2 is a fluid sample taken from a mammal to assess the concentration of nitric oxide within the quantity of mammalian body fluid 2. The mammalian body fluid is preferred to be either saliva, shown in FIG. 1, or urine, shown in FIG. 2, as nitric oxide is typically excreted from either saliva glands or urine. The concentration of nitric oxide can be directly correlated with the production of anandamide within the mammal. The chromatic identifier 3 is a set of discrete pigmentations over a range of concentrations for nitric oxide mixed with the nitric oxide indicator 1. The quantity of nitric oxide indicator 1 is deposited onto the testing medium 4. The testing medium 4 supports the quantity of nitric oxide indicator 1 to allow the quantity of nitric oxide indicator 1 to be submerged within the quantity of mammalian body fluid 2 to react with any nitric oxide present. The testing medium 4 is preferred to be a dipstick. The dipstick allows the user to submerge the nitric oxide indicator 1 in the quantity of mammalian body fluid 2 nitric oxide indicator 1 while preventing direct contact between the user and the quantity of mammalian body fluid 2.

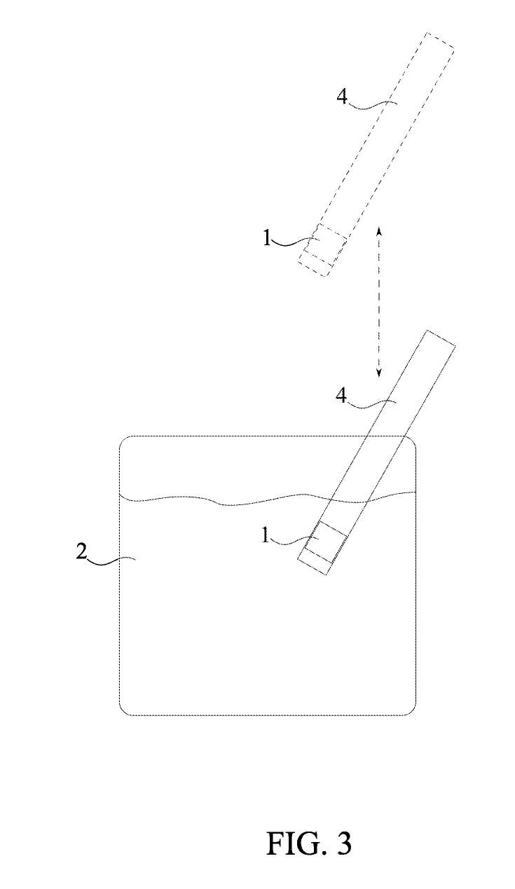

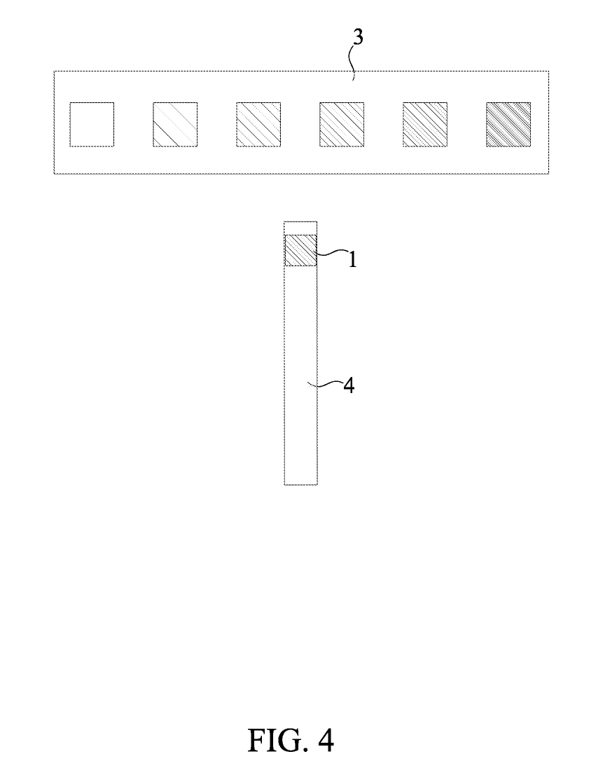

Initially, the testing medium 4 is submerged into the quantity of mammalian body fluid 2, such that the quantity of nitric oxide indicator 1 is exposed to the quantity of mammalian body fluid 2 for a predetermined absorption time, detailed in FIG. 3. The predetermined absorption time is a duration that allows the testing medium 4 to become saturated with the quantity of mammalian body fluid 2. The testing medium 4 is then removed from the quantity of mammalian body fluid 2 after the predetermined absorption time has elapsed to ensure the testing medium 4 is saturated with a portion of the mammalian body fluid. After a predetermined reaction time has elapsed, the quantity of nitric oxide indicator 1 is compared to the chromatic identifier 3, in accordance to FIG. 4 in order to quantify a concentration of nitric oxide. As previously mentioned, the concentration of nitric oxide is directly correlated to the production of anandamide and, therefore, the effectiveness of cannabis consumption is able to be quantified.

Although the invention has been explained in relation to its preferred embodiment, it is to be understood that many other possible modifications and variations can be made without departing from the spirit and scope of the invention as hereinafter claimed.

What is claimed is:

1. A method for standardized testing of anandamide after cannabinoid consumption comprises the steps of:
providing a quantity of nitric oxide indicator, a quantity of mammalian body fluid, a chromatic identifier, and a testing medium, wherein the quantity of nitric oxide indicator is deposited onto the testing medium;
submerging the testing medium into the quantity of mammalian body fluid, such that the quantity of nitric oxide indicator is exposed to the quantity of mammalian body fluid for a predetermined absorption time;
removing the testing medium from the quantity of mammalian body fluid, after the predetermined absorption time has elapsed; and
comparing the quantity of nitric oxide indicator to the chromatic identifier after a predetermined reaction time has elapsed in order to quantify a concentration of nitric oxide.

2. The method for standardized testing of anandamide after cannabinoid consumption, as claimed in claim 1, wherein the testing medium is a dipstick.

3. The method for standardized testing of anandamide after cannabinoid consumption, as claimed in claim 1, wherein the mammalian body fluid is saliva.

4. The method for standardized testing of anandamide after cannabinoid consumption, as claimed in claim 1, wherein the mammalian body fluid is urine.

5. The method for standardized testing of anandamide after cannabinoid consumption, as claimed in claim 1, wherein the quantity of nitric oxide indicator is 4,5-Diaminofluorescein diacetate.

6. A method for standardized testing of anandamide after cannabinoid consumption comprises the steps of:
providing a quantity of nitric oxide indicator, a quantity of mammalian body fluid, a chromatic identifier, and a testing medium, wherein the quantity of nitric oxide indicator is deposited onto the testing medium;
wherein the quantity of nitric oxide indicator is 4,5-Diaminofluorescein diacetate;
submerging the testing medium into the quantity of mammalian body fluid, such that the quantity of nitric oxide indicator is exposed to the quantity of mammalian body fluid for a predetermined absorption time;
removing the testing medium from the quantity of mammalian body fluid, after the predetermined absorption time has elapsed; and
comparing the quantity of nitric oxide indicator to the chromatic identifier after a predetermined reaction time has elapsed in order to quantify a concentration of nitric oxide.

7. The method for standardized testing of anandamide after cannabinoid consumption, as claimed in claim 6, wherein the testing medium is a dipstick.

8. The method for standardized testing of anandamide after cannabinoid consumption, as claimed in claim 6, wherein the mammalian body fluid is saliva.

9. The method for standardized testing of anandamide after cannabinoid consumption, as claimed in claim 6, wherein the mammalian body fluid is urine.
